(19) United States
(12) Patent Application Publication
(10) Pub. No.: US 2005/0187526 A1
(43) Pub. Date: Aug. 25, 2005

(54) Sanitation method utilizing a portable and disposable bidet

(76) Inventor: Matthew Horne, North Chicago, IL (US)

Correspondence Address: WEISS & MOY PC, 4204 NORTH BROWN AVENUE, SCOTTSDALE, AZ, 85251, US

(21) Appl. No.: 10/782,608

(22) Filed: Feb. 19, 2004

(51) Int. Cl. A61M 31/00

(52) U.S. Cl. 604/279

(57) ABSTRACT

A sanitizing method utilizing a portable bidet is disclosed. A portable bidet is provided, consisting of a container having a liquid contained therein, with the liquid being discharged from the container under pressure onto a genital or anal region of a user. The container may be sealed, and may have a nozzle. The container may be deformable, so that pressure is imparted through a squeezing action, or may have a pressurized gas source to create pressure.

### FIELD OF THE INVENTION

This invention relates generally to sanitation methods and, more particularly, to a sanitation method utilizing a portable and disposable bidet.

### BACKGROUND OF THE INVENTION

In most developed countries, flushable toilets are used to carry away human waste, as well as toilet paper that has been used to clean the anal region following defecation, or the genital region following urination. Within some developed countries, the use of a bidet to assist in the cleaning process following defecation or urination is preferred. A bidet is typically located in the bathroom near the toilet.

The use of a bidet can help prevent certain infections, and can help in the treatment of various conditions relating to the anal and genital areas. These include vaginal yeast infections, chronic urinary tract infections (cystitis and urethritis), hemorrhoids, constipation, incontinence, postpartum discharges, vulvodynia, anal pruritis, and anal fissures. The use of a bidet has been particularly recommended for senior citizens, pregnant women, and young children. They are considered to be especially helpful in preserving feminine hygiene.

A bidet is typically a porcelain structure having a bowl similar in design to flush toilets but with a fixed, upwardly oriented water spray nozzle. Hot and cold water valves permit a user to adjust the temperature and flow of water through the nozzle. Thus, a person desiring to utilize a bidet will typically adjust the water flow and temperature, and then position the body over the nozzle so that the flow of water is directed to the desired region or regions. Following washing, a towel, blow dryer, or toilet paper may be used to remove any remaining water on the person's body.

While flush toilets are generally available in most populated areas of most developed countries, there are places where they are generally not available. This includes remote sites, such as camping areas and the like. In addition, in less developed countries, flush toilets may not generally be available. In both developed and less developed countries, even where flush toilets are present, bidets are typically found only in private homes or in hotel bathrooms.

Battery-operated, electronic travel bidets do exist, and these address some of the issues addressed herein. However, they are relatively expensive (for example the NEXWELL travel bidet sells for $70), require batteries, and are somewhat complicated mechanically. Because they need batteries, they may not be convenient for use in locations where batteries are not readily available. As with any electronic device, they are vulnerable to failure. Because they are reusable, they can become contaminated. Finally, because they rely on a local water source, they may not be of benefit in an area where water purity cannot be assured.

A need therefore existed for a portable and disposable sanitizing method that permits a person to experience the benefits of a bidet type of cleansing, in the absence of a bidet. The method should utilize a portable, disposable, one-time use container. It should permit the direction of a flow of water under low pressure to a person's genital or anal region.

The present invention satisfies these needs and provides other, related, advantages.

### SUMMARY OF THE INVENTION

In accordance with one embodiment of the present invention, a method for washing utilizing a portable bidet is disclosed. The method comprises the steps of: providing a portable container having an exit; wherein the container is sized so as to be able to fit within a single human hand during use; providing liquid contained within the container; providing a nozzle coupled to the exit; and permitting a user to force the liquid, under pressure, through the exit and through the nozzle and onto one of an anal and a genital area of a user; wherein the step of permitting a user to force the liquid, under pressure, further comprises the step of providing a deformable the container so that pressure upon the liquid may be imparted by squeezing the container.

In accordance with another embodiment of the present invention, a method for washing utilizing a portable bidet is disclosed. The method comprises the steps of: providing a portable container having an exit; wherein the container is sized so as to be able to fit within a single human hand during use; covering the exit with a seal; providing liquid contained within the container; providing a nozzle coupled to the exit; wherein the nozzle is insertable through the seal; permitting a user to force the liquid, under pressure, through the exit and through the nozzle and onto one of an anal and a genital area of a user; and wherein the step of permitting a user to force the liquid, under pressure, further comprises the step of providing a deformable the container so that pressure upon the liquid may be imparted by squeezing the container.

In accordance with a further embodiment of the present invention, a method for washing utilizing a portable bidet is disclosed. The method comprises the steps of: providing a portable container having an exit; wherein the container is sized so as to be able to fit within a single human hand during use; providing liquid contained within the container; providing a nozzle coupled to the exit; permitting a user to force the liquid, under pressure, through the exit and through the nozzle and onto one of an anal and a genital area of a user; wherein the step of permitting a user to force the liquid, under pressure, further comprises the step of providing a pressurized gas source to propel the liquid under pressure; wherein the pressurized gas source is located in an interior portion of the container.

### BRIEF DESCRIPTION OF THE DRAWINGS

FIG. 1 is a perspective view of a container, adapted to be utilized in connection with a portable sanitizing method consistent with an embodiment of the present invention.

FIG. 2 is a perspective view of a container, adapted to be utilized in connection with a portable sanitizing method consistent with an embodiment of the present invention.

FIG. 3 is a perspective view of a container, adapted to be utilized in connection with a portable sanitizing method consistent with an embodiment of the present invention.

FIG. 4 is a perspective view of a container, adapted to be utilized in connection with a portable sanitizing method consistent with an embodiment of the present invention.

FIG. 5 is a perspective view of a container, adapted to be utilized in connection with a portable sanitizing method consistent with an embodiment of the present invention.

FIG. 6 is a perspective view of a container, adapted to be utilized in connection with a portable sanitizing method consistent with an embodiment of the present invention.

FIG. 7 is a perspective view of an aerosol-type container, adapted to be utilized in connection with a portable sanitizing method consistent with an embodiment of the present invention.

### DETAILED DESCRIPTION OF THE PREFERRED EMBODIMENTS

FIGS. 1-7 illustrate different embodiments of a container utilizable in connection with a portable sanitation method consistent with the present invention. While they differ in their particular configurations and in some of their attributes, they share certain common features.

Thus, each comprises a container that contains a liquid. The liquid is preferably water. It may be desired to treat the water with soap or some other cleaning agent, with a fragrance, or otherwise as desired. Each container permits the liquid to be discharged through an exit in the container. Each permits the liquid to be discharged in a sufficiently focused manner so as to be able to target and cleanse the genital or anal region of a user. Furthermore, each permits the imparting of sufficient pressure on the liquid so as to permit such focused discharge—with the pressure being created either by mechanical force (i.e., the squeezing of the container) or through the use of a gas under pressure. Preferably, each container is disposable. It is also preferred that each container, in order to ensure portability and convenience, be sized as to be capable of being held during use as described herein in a single human hand.

Referring first to FIG. 1, a container 10 is shown. The container 10 is preferably formed of a deformable, plastic type of material. It contains a liquid as herein described. A twistable cap 12 seals the container 10, so as to prevent the liquid from exiting therefrom. This permits the container 10 to be transported in a purse, suitcase, bag or the like, without leaking, until ready for use. In addition to preventing leaking, the seal prevents contamination of the liquid, and thus reduces the risk of infection or other harm following the discharge of the liquid into a body orifice.

A person desiring to use container 10 must first twist and remove cap 12. This unseals the container 10, and exposes an opening through which the liquid is discharged. Because the container 10 is deformable, a person may impart pressure on the liquid by squeezing the container 10, causing the liquid to flow upward or otherwise against gravity, as desired, to clean the desired region. In this configuration, the tapering of the container 10 facilitates the imparting of pressure, and also the focusing of the discharged liquid. As herein illustrated, the container 10 should be utilized a single time only, and thereafter discarded in an appropriate manner.

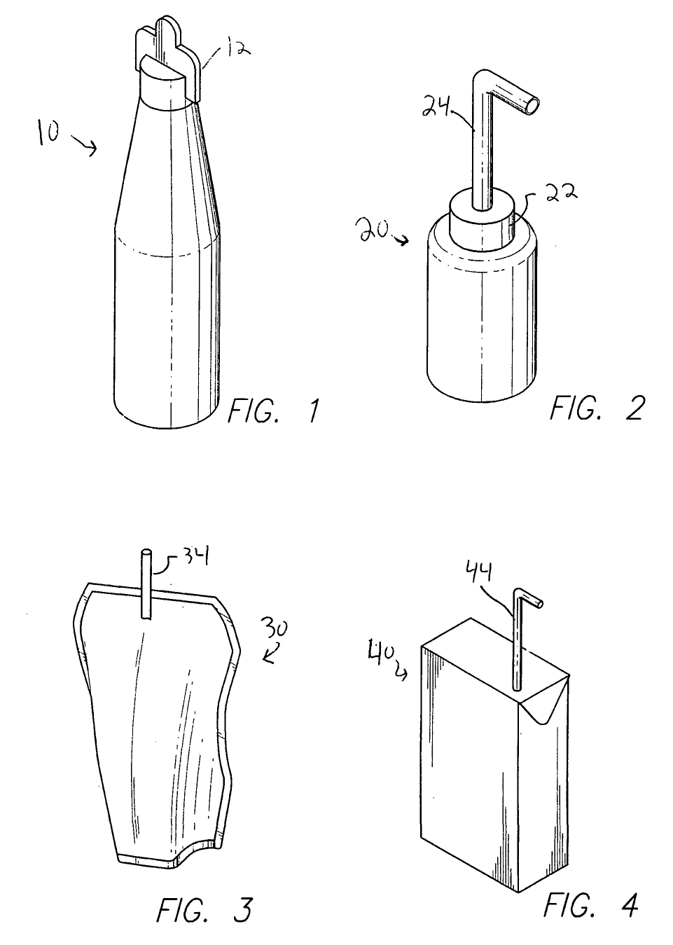

Referring now to FIG. 2, a container 20 is shown. The container 20 is preferably formed of a deformable, plastic type of material. It contains a liquid as herein described. A cap 22 closes the container 20. Preferably, the cap 22 covers a foil or other type of seal (not shown), which prevents the liquid from exiting the container 20 prior to use, and protects the liquid from contamination, for the reasons described above. In this embodiment, a straw-type nozzle 24 is also provided, which is insertable into the cap 22. (In one embodiment, the nozzle 24 and cap 22 may be a one-piece assembly.)

A person desiring to use container 20 must first break the seal. In one embodiment, the cap 22 is removed, the seal removed or broken, and the cap 22 restored. Positioning of the nozzle 24 then permits the discharge of liquid therethrough. In another embodiment, the nozzle 24 has a sharpened lower end, which end may be pushed down through the seal, so as to break the seal and permit the flow of liquid. Because the container 20 is deformable, a person may impart pressure on the liquid by squeezing the container 20, causing the liquid to flow upward or otherwise against gravity, as desired, to clean the desired region.

As shown in FIG. 2, in this embodiment, the nozzle 24 is angled. This facilitates aiming, and can reduce the amount a person may be required to bend in order to properly position the container 20 for use. The use of a relatively narrow straw-type nozzle 24 facilitates the imparting of pressure, and also the focusing of the discharged liquid. As herein illustrated, the container 20 should be utilized a single time only, and thereafter discarded in an appropriate manner.

Referring now to FIG. 3, a container 30 is shown. The container 30 is preferably formed of a deformable, foil type of material formed into a bladder type of configuration. It contains a liquid as herein described. A foil or other type of seal (not shown) closes the container 30 before use, for the reasons and benefits described above. In this embodiment, a straw-type nozzle 34 is also provided, which is insertable into the container 30 through the seal. Preferably, the nozzle 34 has a sharpened lower end, which end may be pushed down through the seal, so as to break the seal and permit the flow of liquid. Prior to use, the nozzle 34 is preferably located in a sealed wrapper so as to limit the risk of contamination, and glued or otherwise associated with the container 30.

Because the container 30 is deformable, a person may through a squeezing action impart pressure on the liquid, causing it to flow upward or otherwise against gravity, as desired, to clean the desired region. In this embodiment, as shown in FIG. 3, the nozzle 34 is straight. As herein illustrated, the container 30 should be utilized a single time only, and thereafter discarded in an appropriate manner.

Referring now to FIG. 4, a container 40 is shown. The container 40 is preferably formed of a deformable, pulp type of material formed into a box type of configuration. It contains a liquid as herein described. A foil or other type of seal (not shown) closes the container 40 before use, for the reasons and benefits described above. In this embodiment, a straw-type nozzle 44 is also provided, which is insertable into the container 40 through the seal. Preferably, the nozzle 44 has a sharpened lower end, which end may be pushed down through the seal, so as to break the seal and permit the flow of liquid. Prior to use, the nozzle 44 is preferably located in a sealed wrapper so as to limit the risk of contamination, and glued or otherwise associated with the container 40.

Because the container 40 is deformable, a person may by squeezing impart pressure on the liquid, causing it to flow upward or otherwise against gravity, as desired, to clean the desired region. As shown in FIG. 4, in this embodiment, the nozzle 44 is angled. This facilitates aiming, and can reduce the amount a person may be required to bend in order to properly position the container 40 for use. The use of a relatively narrow straw-type nozzle 44 facilitates the imparting of pressure, and also the focusing of the discharged liquid. As herein illustrated, the container 40 should be utilized a single time only, and thereafter discarded in an appropriate manner.

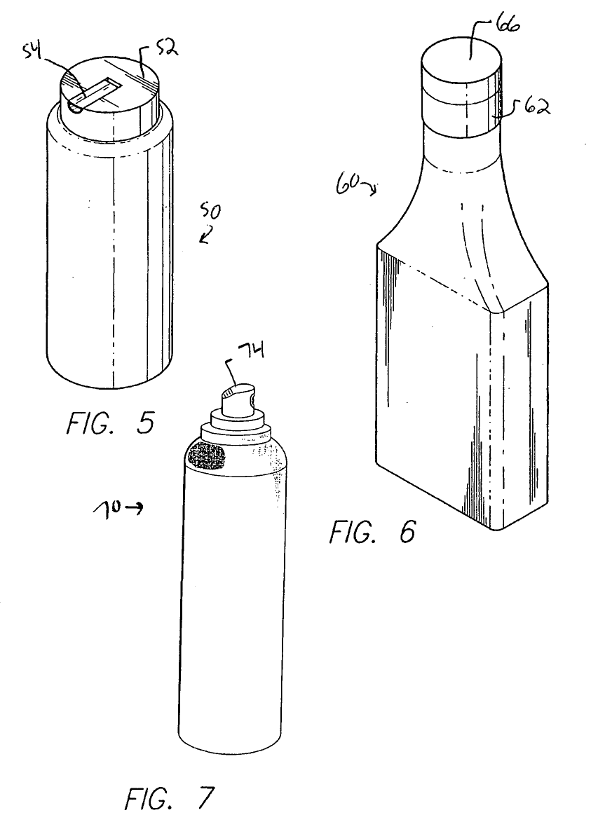

Referring now to FIG. 5, a container 50 is shown. The container 50 is preferably formed of a deformable, plastic type of material. It contains a liquid as herein described. A cap 52 closes the container 50. Preferably, the cap 52 covers a foil or other type of seal (not shown), which prevents the liquid from exiting the container 50 prior to use, and protects the liquid from contamination, for the reasons described above. In this embodiment, a flip-up nozzle 54 is also provided, which is coupled to the cap 52.

A person desiring to use container 50 must first break the seal. In one embodiment, the cap 52 is removed, the seal removed or broken, and the cap 52 restored. Flipping up of the nozzle 54 then permits the discharge of liquid therethrough. Because the container 50 is deformable, a person may through a squeezing action impart pressure on the liquid, causing it to flow upward or otherwise against gravity, as desired, to clean the desired region. As herein illustrated, the container 50 should be utilized a single time only, and thereafter discarded in an appropriate manner.

Referring now to FIG. 6, a container 60 is shown. The container 60 is preferably formed of a deformable, plastic type of material, and tapers upward. It contains a liquid as herein described. A cap 62 having a lid 66 closes the container 60. The lid 66 preferably conceals and covers an opening (not shown) in the cap 62. Preferably, the cap 62 covers a foil or other type of seal (not shown), which prevents the liquid from exiting the container 60 prior to use, and protects the liquid from contamination, for the reasons described above.

A person desiring to use container 60 must first break the seal. In one embodiment, the cap 62 is removed, the seal removed or broken, and the cap 62 restored. Flipping up of the lid 66 then permits the discharge of liquid from the container 60 through the opening (not shown) in the cap 62.

Because the container 60 is deformable, a person may by squeezing impart pressure on the liquid, causing it to flow upward or otherwise against gravity, as desired, to clean the desired region. In this configuration, the tapering of the container 60 facilitates the imparting of pressure, and also the focusing of the discharged liquid. As herein illustrated, the container 60 should be utilized a single time only, and thereafter discarded in an appropriate manner.

Referring now to FIG. 7, a container 70 is shown. The container 70, in this embodiment, is preferably aerosol type. Liquid is contained therein, under pressure, and may be discharged via spray nozzle 74. A person desiring to use container 70 may first be required to shake the container 70 in order to increase the effectiveness of the propellant located therein. The nozzle 74 is then aimed, and the nozzle 74 depressed. Liquid is then discharged under pressure through nozzle 74 toward the desired area. It may be desired to further provide a straw-type second nozzle (not shown), insertable into the nozzle 74, for purposes of increasing accuracy and perhaps also providing increased pressure.

It should be noted that FIG. 7 merely shows one exemplary embodiment of a container having liquid located therein under pressure. Other means may be utilized to create such pressure, including for example a separate gas cartridge that may be coupled to the container for use.

While the invention has been particularly shown and described with reference to preferred embodiments thereof, it will be understood by those skilled in the art that the foregoing and other changes in form and details may be made therein without departing from the spirit and scope of the invention.

For example, it may be desired to provide a brush or sponge attachment to a nozzle or container, to assist in the cleaning process. In addition, it may be desired to provide shielding for the exposed nozzle end, so as to prevent its accidental and possible damaging insertion into an anal or vaginal cavity. While, generally, it is preferred that the containers be designed for one-time use and then discarded, and further that they be filled during a manufacturing process and then sealed, it may be desirable to provide a container as herein described that is refillable by the user (and perhaps also initially fillable by the user) and that may be re-used. Additional such variations may be possible, provided that the container utilized is portable and self-contained, and has a liquid therein which may be discharged therefrom under pressure toward an anal or genital region.

1. A method for washing utilizing a portable bidet comprising the steps of:
providing a portable container having an exit;
wherein said container is sized so as to be able to fit within a single human hand during use;
providing liquid contained within said container;
providing a nozzle coupled to said exit; and
permitting a user to force said liquid, under pressure, through said exit and through said nozzle and onto one of an anal and a genital area of a user;
wherein said step of permitting a user to force said liquid, under pressure, further comprises the step of providing a deformable said container so that pressure upon said liquid may be imparted by squeezing said container.

2. The method of claim 1 wherein said container is disposable.

3. The method of claim 1 wherein said container is primarily comprised of plastic.

4. The method of claim 1 wherein said container is primarily comprised of wood pulp.

5. The method of claim 1 wherein said container is bladder-type.

6. The method of claim 1 wherein said container is upwardly tapered.

7. The method of claim 1 wherein said nozzle is angled so as to facilitate aiming of said liquid.

8. A method for washing utilizing a portable bidet comprising the steps of:
providing a portable container having an exit;
wherein said container is sized so as to be able to fit within a single human hand during use;
covering said exit with a seal;
providing liquid contained within said container;
providing a nozzle coupled to said exit;
wherein said nozzle is insertable through said seal;
permitting a user to force said liquid, under pressure, through said exit and through said nozzle and onto one of an anal and a genital area of a user; and
wherein said step of permitting a user to force said liquid, under pressure, further comprises the step of providing a deformable said container so that pressure upon said liquid may be imparted by squeezing said container.

9. The method of claim 8 wherein said nozzle is affixed to said container over said seal when not in use.

10. The method of claim 8 wherein said nozzle is angled so as to facilitate aiming of said liquid.

11. The method of claim 8 wherein said container is disposable.

12. The method of claim 8 wherein said container is primarily comprised of plastic.

13. The method of claim 8 wherein said container is primarily comprised of wood pulp.

14. The method of claim 8 wherein said container is bladder-type.

15. The method of claim 8 wherein said container is upwardly tapered.

16. A method for washing utilizing a portable bidet comprising the steps of:
providing a portable container having an exit;
wherein said container is sized so as to be able to fit within a single human hand during use;
providing liquid contained within said container;
providing a nozzle coupled to said exit;
permitting a user to force said liquid, under pressure, through said exit and through said nozzle and onto one of an anal and a genital area of a user;
wherein said step of permitting a user to force said liquid, under pressure, further comprises the step of providing a pressurized gas source to propel said liquid under pressure;
wherein said pressurized gas source is located in an interior portion of said container.

17. The method of claim 16 wherein said container is an aerosol-type container.
